(19) United States
(12) Patent Application Publication
(10) Pub. No.: US 2002/0049174 A1
(43) Pub. Date: Apr. 25, 2002

(54) Structured antisense nucleic acid molecules

(76) Inventors: Martin Stocks, Cambridge (GB); Terrence Rabbitts, Cambridge (GB)

Correspondence Address: Kathleen Madden Williams, Palmer & Dodge, LLP, One Beacon Street, Boston, MA, 02108, US

(21) Appl. No.: 09/742,675

(22) Filed: Dec. 20, 2000

Related U.S. Application Data
(60) Provisional application No. 60/090,867, filed on Jun. 26, 1998.

(30) Foreign Application Priority Data
Jun. 23, 1999 (GB) GB99/01956
Jun. 23, 1998 (GB) 9813531.2

(51) Int. Cl. A61K 48/00; C12Q 1/68; C07H 21/04

(52) U.S. Cl. 514/44; 536/23.2; 435/6

(57) ABSTRACT

The present invention relates to an antisense nucleic acid molecule comprising a first region and a second region, both of which are complementary to a target nucleic acid molecule, and wherein the first region is available for hybridization and the second region is temporarily masked. The antisense molecules of the invention display increased specificity and stability of binding.

The present invention relates to antisense nucleic acid molecules. Specifically, the invention relates to structured antisense RNA molecules in which a part of the molecule is temporarily masked.

Antisense oligonucleotides spanning regions of mRNA responsible for the production of undesired gene products have been used extensively in attempts to downregulate the gene products. Antisense molecules have been targeted at the coding sequence of mRNA, the translation start site, 3′ and 5′ untranslated regions, intron/exon splice junctions and practically every other part of the RNA molecule.

A significant number of haematopoietic tumours and tumours of mesenchymal origin, possibly also including other tumours such as those of epithelial origin, possess specific chromosomal translocations which are thought to be responsible for the onset and maintenance of malignancy (1). Frequently these translocations result in the fusion of two genes. As these entities arise as somatic events in cells which appear as tumours, such fusion genes or gene products present interesting possible targets for therapeutic intervention (2). The paradigm of a gene fusion resulting from a consistently observed chromosomal translocation is the BCR-ABL fusion found in Philadelphia-positive chronic myelogenous leukaemia (CML) and acute lymphocytic leukaemia (ALL) resulting from t(9;22)(q34;q11) translocations (3). Many studies have been carried out using various methods to functionally delete the BCR-ABL gene product, including antisense RNA techniques (4, 5, 6, 7, 8, 9).

One major problem with antisense approaches in general is the conflicting requirements for the stability of the antisense binding and its specificity for the target RNA. This problem is particularly acute in the case of chromosomal translocation targets such as BCR-ABL, since the targeting must be conducted in the presence of the normal BCR and ABL mRNA species (10). In principle, shorter antisense molecules are more likely to bind to the fusion mRNA specifically but longer antisense molecules are necessary to bind to fusion mRNA targets with sufficient stability (i.e. with slower off rates) to affect function (11, 12).

Investigators have focused their efforts on developing more stable, modified antisense nucleic acids which, through chemical modification, are capable of binding more tightly to a given length of RNA sequence, and on targeting sites in the RNA which are more sensitive to disruption. There remains a need, however, for a generally applicable antisense technique which permits specific targeting of antisense oligonucleotides which are capable of sufficiently stable binding to influence mRNA translation.

### SUMMARY OF THE INVENTION

According to a first aspect of the present invention, there is provided an antisense nucleic acid molecule comprising a first region and a second region, both of which are complementary to a target nucleic acid molecule, and wherein the first region is available for hybridisation and the second region is temporarily masked.

In a second aspect, the invention provides a method for modulating the expression of a gene product encoded by a target nucleic acid by hybridisation with an antisense nucleic acid molecule, comprising the steps of:

(a) preparing an antisense nucleic acid molecule according to the first aspect of the invention;

(b) hybridising the antisense molecule to the target nucleic acid such that the first region of the antisense molecule binds to its complementary sequence in the target nucleic acid; and

(c) continuing the hybridisation, such that the second region of the antisense molecule hybridises to its complementary sequence in the target nucleic acid.

### BRIEF DESCRIPTION OF THE FIGURES

FIG. 1. Design of the hAS series of structured antisense molecules.

A. The intended structure of the hAS series of RNAs: A diagram of the hAS190α form is shown. Antisense residues are shown in black (bold line), structural residues in greyscale or black (thin line). The targeting region (boxed) is a single stranded region between stem loops I and II. The antisense molecule is drawn 5′→3′. The relationship of the targeting loop to the BCR-ABL mRNA is shown. The two forms of the hAS190 molecule, designated α and β, differ only in the descending strand of stem/loop II and the differences present in the loop of hAS190β form are shown in brackets.

B. T1 ribonuclease mapping of the hAS190α transcript. Lane 1 is a T1 ribonuclease digest of the denatured RNA showing the location of each G residue. Lane 2 is a T1 ribonuclease digest of the native transcript. Lane 3 is a partial alkaline hydrolysis ladder. The positions of the regions of interest (e.g. antisense loop) are indicated.

C. A model for the interaction of an hAS antisense with its target mRNA. 1. The antisense makes initial contact via the targeting loop. 2. Breathing of the open ends of the stems allows for further interaction of the antisense sequence with the target. As the antisense/target hybrids have a much lower free energy than the stem/loop structures of the hAS molecule, this process is driven forward 3. The stem/loops are completely unravelled and the antisense region is stably hybridised to its target along its full length.

FIG. 2. The interaction of hAS190 RNAs with the p190 target RNA.

A. Band-shift gel demonstrating the interaction of hAS190α with p190 over 120 minutes, as indicated.

B. Band-shift gel showing the presence of antisense and target RNA molecules in the hybrid RNAs from FIG. 2A. Lane 1: Both antisense and sense RNAs radio-labelled. Lane 2: Radio-labelled antisense and unlabelled target sense RNA. Lane 3: Radio-labelled target sense RNA and unlabelled antisense. The position of uncomplexed antisense or sense RNAs are arrowed and the hybrid molecules are also indicated.

FIG. 3. The specificity of interaction of the hAS molecules. Band-shift gel showing specific binding of labelled antisense molecule to intended target RNA. Labelled antisense is mixed with unlabelled RNA, as indicated, and run on a 4M urea denaturing gel.

FIG. 4. Blocking of the hAS/target interaction by oligonucleotide.

A. Band-shift assay showing binding of antisense RNA to its target in the presence of oligonucleotides complementary to the target sequence binding to the stem/loop of the antisense molecule.

B. A schematic version of hAS190α showing the regions complementary to the blocking oligonucleotides: 1, oligo 1; 2, oligo 2 and 3, oligo 3.

FIG. 5. Maps of Antisense vectors.

Restriction maps of vectors pUN-1, pUN-1.Tal and pUN-1.SQ2 are shown. The human U6 promoter transcript is a 328 bp Pfu fragment amplified from genomic DNA, ending at base +1 of U6 snRNA. The PolIII terminator is a synthetic oligonucleotide containing the ′TTTTT′ RNA polIII termination signal.

FIG. 6. Inhibition of BCR-ABL expression in vivo.

Histogram showing data derived from a western blot measured by densitometry of the western ECL signal. Protein levels observed in cells 16 hours post-transfection are shown for the transfected p190 BCR-ABL and for endogenous BCR. Cells are cotransformed with p190 BCR-ABL, plus one of the following (see FIG. 5):

pUN-1 is the empty U6 promoter-based expression vector.

pUN1-Tal is the U6 vector expressing a Tal-1 antisense RNA

pUN1-SQ2 is the U6 vector expressing hAS-p190α.

FIG. 7. Specificity of BCR-ABL expression in vivo.

Histogram showing control data derived from a western blot measured by densitometry of the western ECL signal as for FIG. 6. Protein levels observed in cells 16 hours post-transfection are shown for the transfected p210 BCR-ABL and for endogenous c-ABL. Cells are cotransformed with p210 BCR-ABL, plus one of the vectors as for FIG. 6.

### DETAILED DESCRIPTION OF THE INVENTION

Definitions

Antisense In accordance with the present invention, the term “antisense” is used to describe the reagents and the methods employed in techniques known in the art by this name. Particularly, the term refers to the use of nucleic acid molecules complementary to nucleic acids present in organisms, especially RNAs, to modulate the expression of specific genes. In a preferred embodiment, the term refers to the use of nucleic acids complementary to mRNA molecules in order to modulate the processing thereof, especially their translation. Preferably, the modulation is directed at down-regulating gene expression, for example through prevention of mRNA translation or by degradation of the mRNA, such as for example by RNase H targeting. Accordingly, in the broadest sense, an antisense molecule is simply a molecule which is at least partly complementary to a target nucleic acid. Preferably, the nucleic acid is a mRNA encoding a specific gene product. Advantageously, an antisense molecule is a molecule which is at least partly complementary to a target nucleic acid and which, moreover, is effective in modulating gene expression through an antisense mechanism.

Region A “region”, as used herein for example in “first region” and “second region”, is a part of a molecule. In the case of a nucleic acid molecule, a region is a stretch of bases, preferably a contiguous stretch of bases.

Nucleic acid As used herein, “nucleic acid” refers to any natural nucleic acid, including RNA and DNA as well as synthetic nucleic acid comprising modified or synthetic bases, and mixtures of modified or synthetic bases with natural bases. Such modified and/or synthetic bases may be referred to as derivatives of DNA or RNA. Preferably, “nucleic acid” refers to RNA, such that one or both of the target nucleic acid molecule and the antisense nucleic acid molecule are RNA molecules.

Masked A feature of the present invention is that the antisense nucleic acid molecule comprises a region complementary to the target nucleic acid which is temporarily masked. This means that it is unavailable for hybridisation, for example by reason of its being associated with a masking group. Preferably, a region is masked by being hybridised to a further nucleic acid region at least partly complementary thereto, such that it is present as double-stranded nucleic acid and thus unavailable for hybridisation. For example, the second region may be comprised in a hairpin loop or stem/loop structure. These structures, in turn, are inverted repeats in a nucleic acid molecule which allow a part of the molecule to assume a double-stranded conformation by intramolecular hybridisation of the repeats. Often, there is a “loop” at the end of the stem or hairpin, consisting of those bases linking the inverted repeats which cannot hybridise together. In the context of the present invention, the size and/or structure of a loop is not important. For example, however, a loop may consist of between 3 and 10 bases.

Target Nucleic Acid Antisense molecules according to the present invention may be used to modulate the expression of substantially any target nucleic acid. Thus, antisense molecules may be used to target genomic or episomal DNA or RNA, whether endogenous to the cell or heterologous, such as for example viral DNA or RNA. Preferably, however, the antisense molecule is used to target RNA, especially mRNA or pre-mRNA, but also tRNA and other RNA forms.

Specific sequence The first region of the antisense molecules according to the invention are preferably complementary to a specific sequence in the target nucleic acid molecule. By this expression, it is intended that the part of the target nucleic acid molecule which is complementary to the first region is of such a sequence that it permits the binding of the antisense molecule specifically to the target molecule whilst avoiding binding to similar, non-target molecules. Preferably, because the antisense molecules of the invention rely on two separate regions to bind to the target, the specific sequence targeted by the first region should be sufficiently unique to prevent binding of the antisense molecule to non-target molecules which possess sequences complementary to the second region of the antisense molecule.

Destabilising elements may be incorporated in the antisense molecules according to the invention in order to favour unmasking thereof and hybridisation of the second region to the target sequence. In particular, where the second region is masked through incorporation into a stem/loop or hairpin structure, base pair mismatches, G-U base pairings and incorporation of extra bases in one strand in order to cause bulging may be used to induce destabilisation. The aim is to render the second region/target hybrid more stable than the second region in its masked state, thus thermodynamically favouring the formation of the hybrid.

### DESCRIPTION OF SPECIFIC EMBODIMENTS

The concept of the approach embodied in the present invention is that an antisense molecule should initially interact with its cognate fusion mRNA only via a first, short targeting region. After this reaction, unmasking of a second, longer region allows further interaction with increasing lengths of the fusion mRNA.

It has been observed, in studying the performance of antisense nucleic acid molecules, that under in vivo conditions the level of non-specific binding observed with long antisense molecules is sufficient to influence the specificity of a reaction, leading to a lack of specificity in any observed antisense effect. It is possible that, under in vivo conditions, a longer sequence is capable of supporting sufficient miss-matches or loopouts in base pairing to facilitate non-specific hybridisation.

Short antisense molecules, although capable of only binding to very specific sequences, do not bind with sufficient stability to induce an antisense effect. Moreover, they may bind at more than one position in the genome, and thus their binding, even if specific, will not be unique.

The present invention overcomes all of these problems by providing an antisense molecule which binds to its target via a two-step process, in which both steps are required. In the first step, referred to herein as the nucleation step, the molecule binds to a short sequence in its target, known as the nucleation site. This binding event, however, is of insufficient stability to promote any antisense effect and will be transitory unless the second step is also enabled.

The second step involves the binding of a second region of the antisense molecule, previously masked and thus unavailable for hybridisation, to a sequence on the target nucleic acid adjacent to the nucleation site. The binding of the second region imparts the antisense-target hybrid with sufficient stability to promote an antisense effect.

The approach therefore combines the advantages of a short initial targeting region, which avoids non-specific targeting, with those of stability and ability to bind unique sequences associated only with longer antisense molecules.

For instance, in the case of non-target molecules having a sequence complementary to the first region of the antisense molecule but not to the second, the nucleation of the targeting region will occur but will not support the propagation of hybridisation of the second region. Thus, the interaction will be transitory because the first region of the antisense molecule is too short to bind stably to the target nucleic acid. The antisense molecules of the invention accordingly do not bind to non-target molecules, even if the first region of the antisense molecule has an exact complement in the said non-target molecule.

Non-specific hybridisation between the second region and non-target molecules is prevented because the antisense molecule will not nucleate on non-target molecules which lack complementarity with the first region. Even if the second region of antisense is itself complementary to non-target molecules, provided that said non-target molecules do not also comprise a sequence complementary to the first region the nucleation reaction is prevented from occurring. In the latter case, because the second region is unavailable for hybridisation in the antisense molecule and thus cannot itself initiate a hybridisation reaction, any effect on non-target nucleic acids is precluded.

The first region of the antisense molecule of the invention is insufficiently long to provide stable binding by itself. Such a sequence is preferably between 3 and 18, preferably between 5 and 12 and advantageously between 7 and 10 bases in length.

The second region of the antisense molecule of the invention is long enough to provide, optionally in combination with the first region, a hybrid with the target nucleic acid which is both sufficiently stable to mediate a specific antisense effect and potentially unique in the genome of the cell or organism which contains the target nucleic acid. Preferably, the second region is no longer than 100, advantageously between 20 and 70 and preferably between 25 and 45 bases in length.

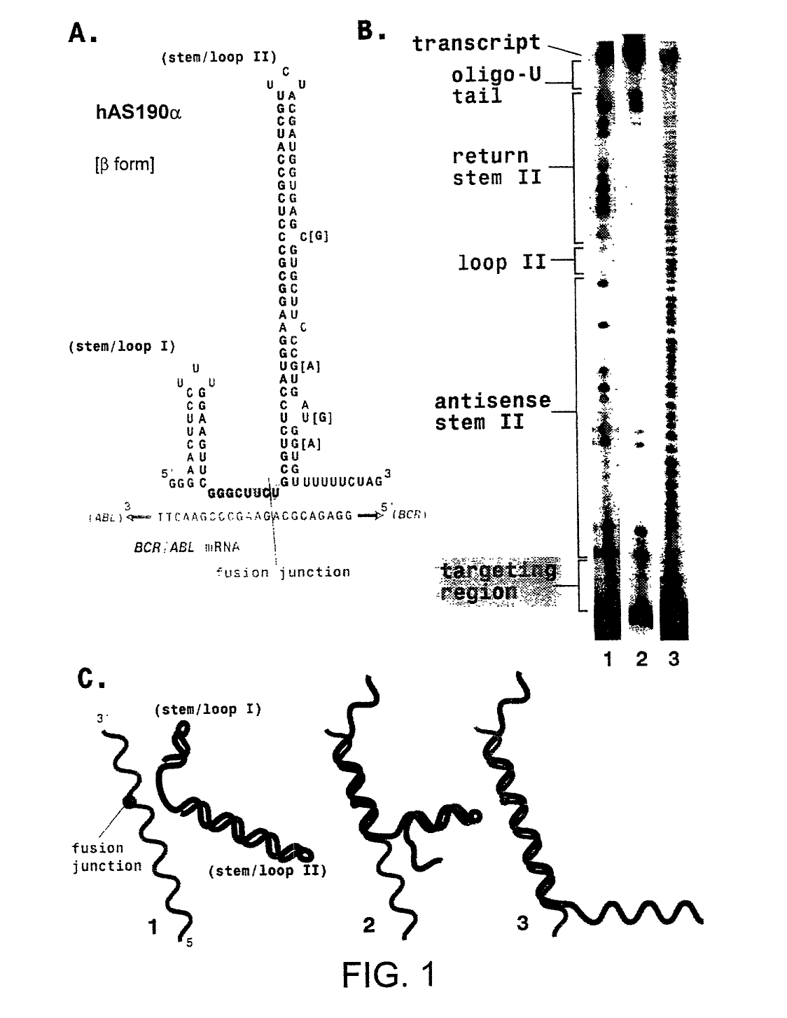

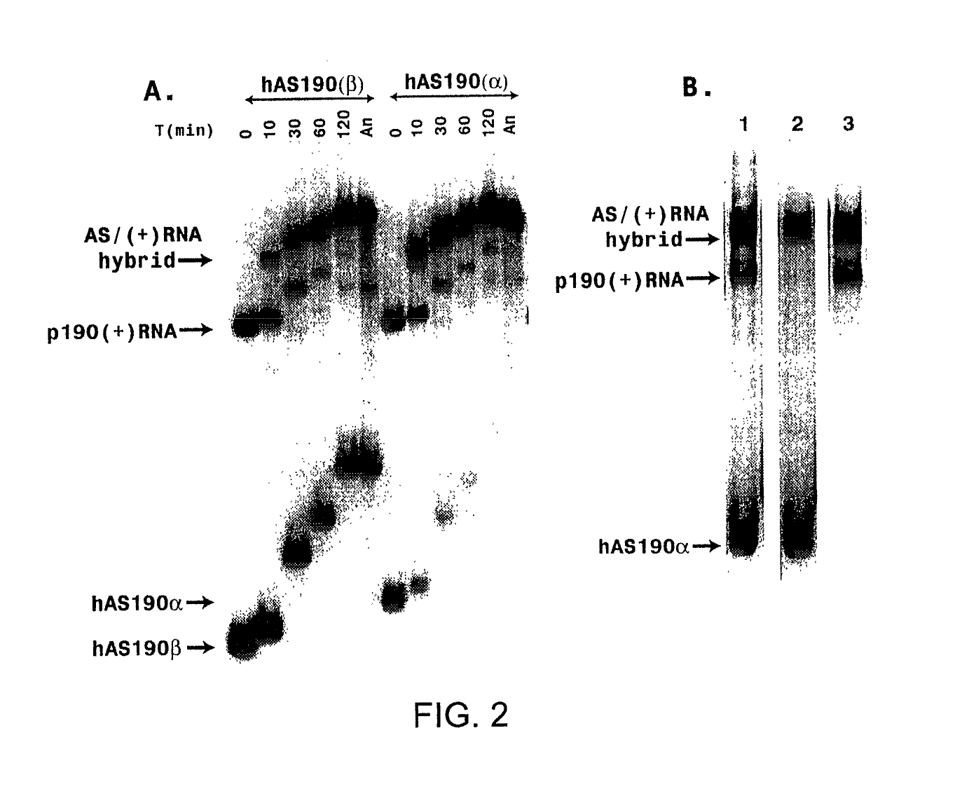

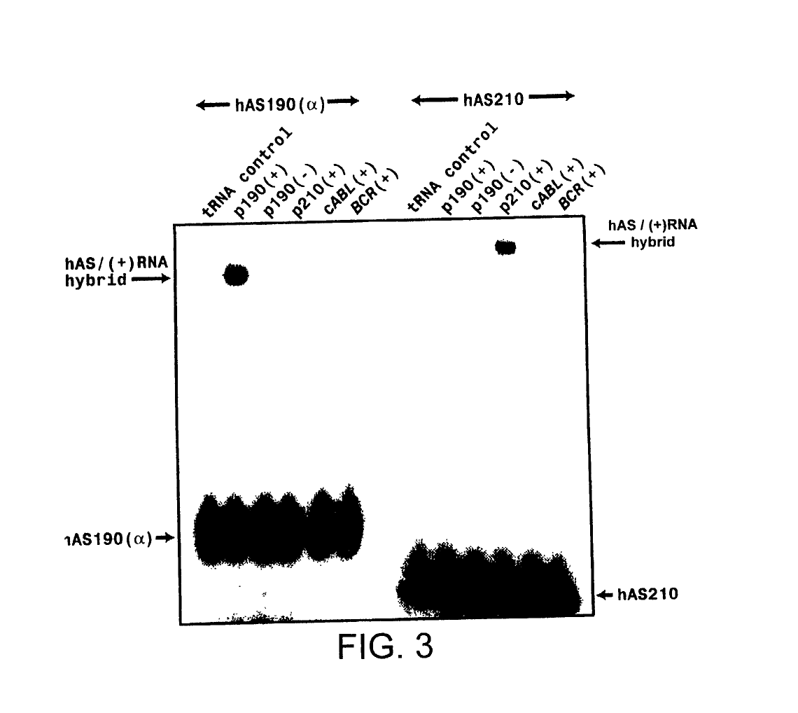

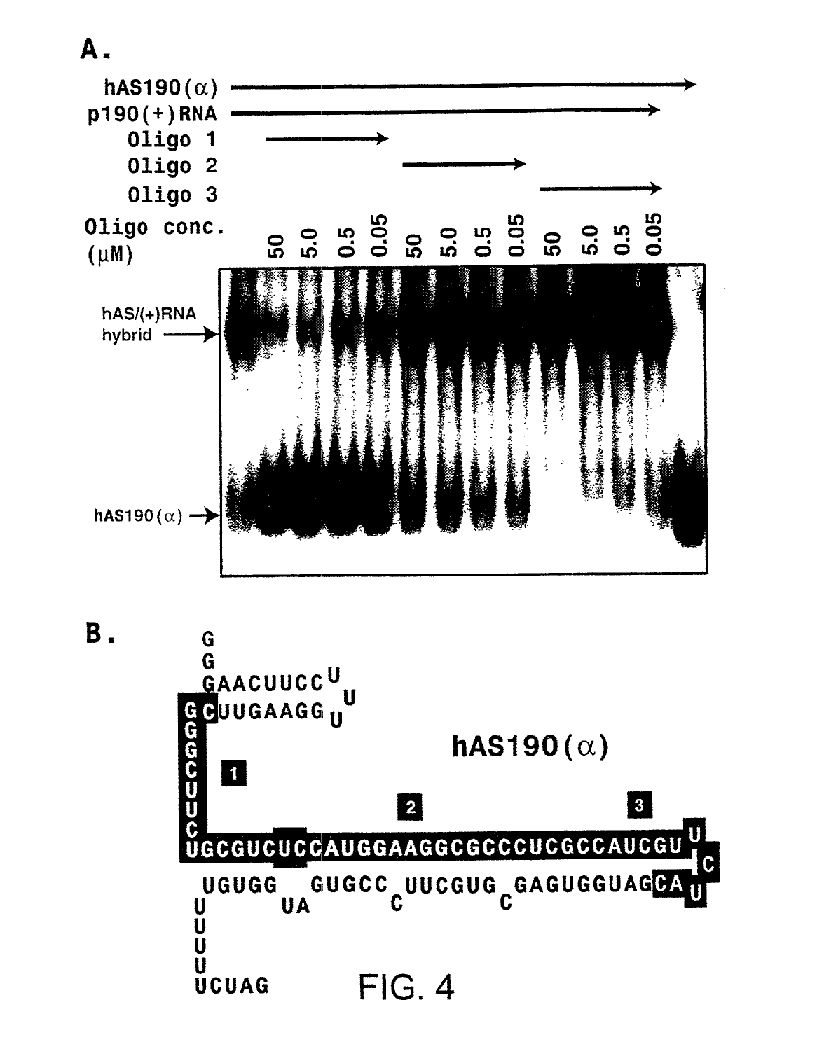

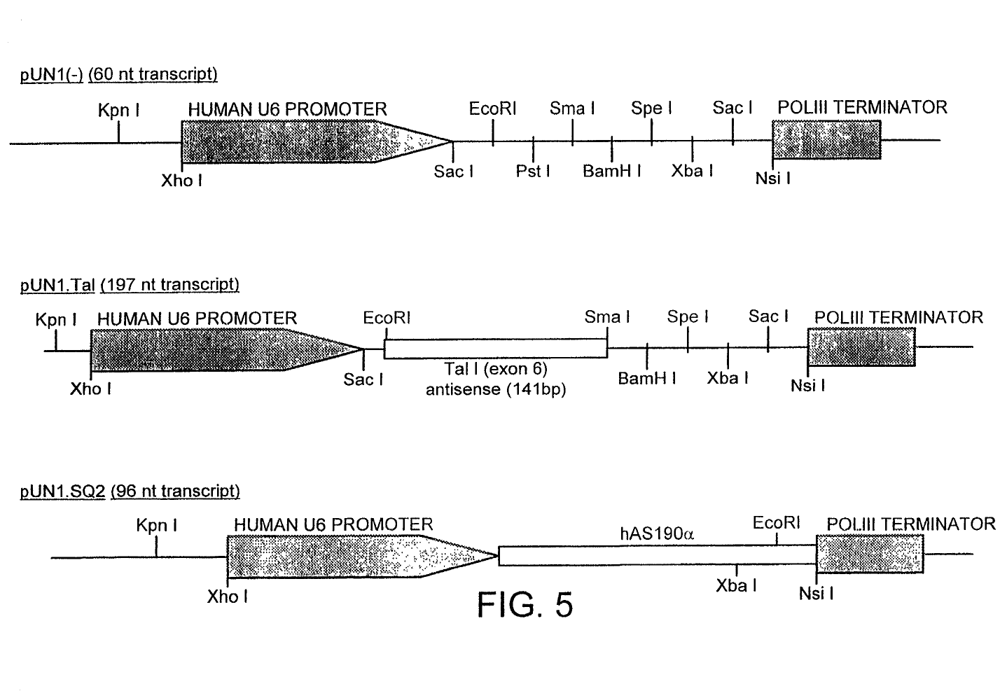

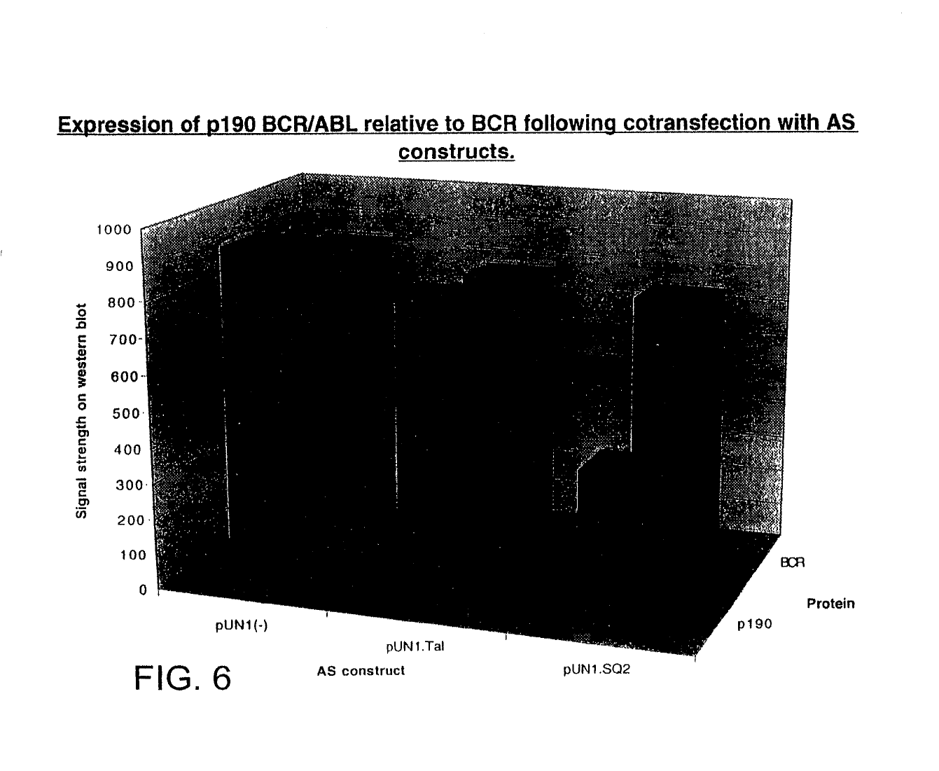

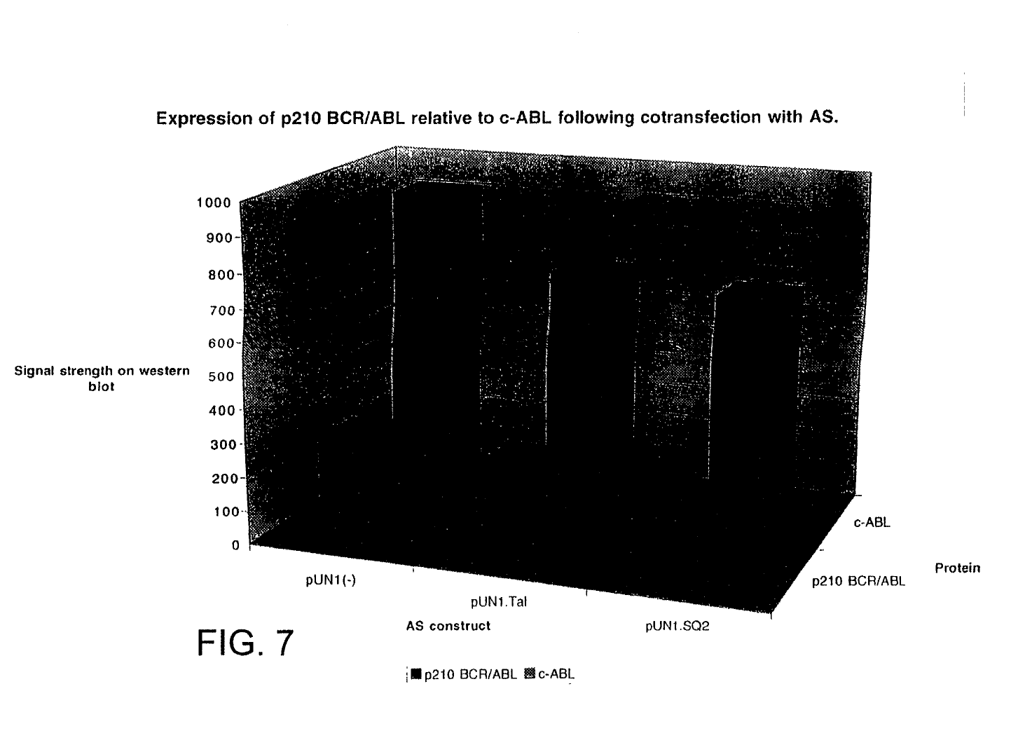

Preferably, masking is achieved by incorporating the second region of the antisense molecule in a stem/loop structure. Molecules incorporating such a structure may be termed “structured antisense molecules” (hAS). In the case of structured antisense molecules, after the nucleation reaction “breathing” in the stem/loop allows the interaction of the second region with the target nucleic acid, resulting in the propagation of a wave of unmasking with the simultaneous hybridisation of the antisense sequence along the target molecule (illustrated in FIG. 1C.).

The stem/loop of the hAS molecule preferably contains destabilising elements (bulges, mismatches, G-U pairs) to render its unwinding, and the association with the target nucleic acid molecule, energetically highly favourable.

Alternatives to stem/loop structures will present themselves to persons skilled in the art. Thus, for example, the second region of the antisense RNA molecule could be complexed with a separate nucleic acid molecule which becomes dissociated from the antisense molecule on binding to the target nucleic acid. In other embodiments, chemical blocking groups may be employed to prevent hybridisation except under the energetically favourable conditions created after the nucleation reaction with the target nucleic acid.

Specific sequences suitable for use as nucleation sites may be found by analysis of desired target sequences. For example, suitable nucleation sites may be located at the junctions of aberrant sequences created by chromosomal translocations, at the sites of mutations which occur in aberrant genes, in regions of nucleic acids deriving from heterologous organisms, such as pathogens, and the like.

As set forth above, it is not essential for the nucleation sequence to be unique in the in vivo system in which the reaction is performed to achieve specificity. The reliance of the method of the invention on two hybridisation reactions provides that the absence of a sequence complementary to the second region of the antisense molecule at or near the site of nucleation will prevent stable binding of the antisense molecule.

Preferably, therefore, the sequences in the target nucleic acid molecule which are complementary to the first and second regions of the antisense nucleic acid molecule are contiguous, or closely juxtaposed. By “closely juxtaposed”, it is intended to indicate that intervening sequences may be present. Where this is the case, the antisense molecule will be arranged so as to allow physical interaction of the first and second regions with their respective complementary sequences.

In a further aspect, the invention relates to a method for hybridising an antisense nucleic acid molecule to a target nucleic acid, comprising the steps of:

(a) preparing an antisense nucleic acid molecule according to the first aspect of the invention;

(b) hybridising the antisense molecule to the target nucleic acid such that the first region of the antisense molecule binds to its complementary sequence in the target nucleic acid; and

(c) continuing the hybridisation, such that the second region of the antisense molecule hybridises to its complementary sequence in the target nucleic acid.

Preferably, the method of the invention is useful for modulating gene expression in vitro or in vivo by an antisense mechanism.

However, the invention may also be applied to any nucleic acid binding requirement. Thus, molecules in accordance with the present invention may be used for designing novel nucleic acid binding proteins such as transcription factors or restriction enzymes. An antisense molecule according to the present invention may be fused to the transcriptional activation domain of a transcription factor, to obtain a novel specificity, or to a nucleic acid cleavage domain of a restriction enzyme.

In a further aspect, the present invention provides a vector suitable for expression of a new nucleic acid sequence including an RNA molecule according to the invention. For example, the vector according to the invention may be suitable for use in gene therapy, delivering RNA molecules according to the invention to sites of need in a patient. Moreover, vectors may be used for amplification of nucleic acids encoding RNA molecules according to the invention, in bacterial, mammalian, insect or other host cells.

A vector according to the invention may be prepared according to the techniques known in the art and familiar to the skilled artisan. For example, nucleic acids encoding structured antisense molecules according to the invention may be incorporated into vectors for further manipulation. As used herein, vector (or plasmid) refers to discrete elements that are used to introduce heterologous DNA into cells for either expression or replication thereof. Selection and use of such vehicles are well within the skill of the artisan. Many vectors are available, and selection of appropriate vector will depend on the intended use of the vector, i.e. whether it is to be used for DNA amplification or for DNA expression, the size of the DNA to be inserted into the vector, and the host cell to be transformed with the vector. Each vector contains various components depending on its function (amplification of DNA or expression of DNA) and the host cell for which it is compatible. The vector components generally include, but are not limited to, one or more of the following: an origin of replication, one or more marker genes, an enhancer element, a promoter, a transcription termination sequence and a signal sequence.

Both expression and cloning vectors generally contain nucleic acid sequence that enable the vector to replicate in one or more selected host cells. Typically in cloning vectors, this sequence is one that enables the vector to replicate independently of the host chromosomal DNA, and includes origins of replication or autonomously replicating sequences. Such sequences are well known for a variety of bacteria, yeast and viruses. The origin of replication from the plasmid pBR322 is suitable for most Gram-negative bacteria, the 2μ plasmid origin is suitable for yeast, and various viral origins (e.g. SV 40, polyoma, adenovirus) are useful for cloning vectors in mammalian cells. Generally, an origin of replication component is not needed for mammalian expression vectors unless these are used in mammalian cells competent for high level DNA replication, such as COS cells.

Most expression vectors are shuttle vectors, i.e. they are capable of replication in at least one class of organisms but can be transfected into another class of organisms for expression. For example, a vector is cloned in E. coli and then the same vector is transfected into yeast or mammalian cells even though it is not capable of replicating independently of the host cell chromosome. DNA may also be replicated by insertion into the host genome. However, the recovery of genomic DNA encoding structured antisense molecules according to the invention is more complex than that of exogenously replicated vector because restriction enzyme digestion is required to excise the nucleic acid encoding the RNA according to the invention. DNA can moreover be amplified by PCR and be directly transfected into the host cells without any replication component.

Advantageously, an expression and cloning vector may contain a selection gene also referred to as selectable marker. This gene encodes a protein necessary for the survival or growth of transformed host cells grown in a selective culture medium. Host cells not transformed with the vector containing the selection gene will not survive in the culture medium. Typical selection genes encode proteins that confer resistance to antibiotics and other toxins, e.g. ampicillin, neomycin, methotrexate or tetracycline, complement auxotrophic deficiencies, or supply critical nutrients not available from complex media.

As to a selective gene marker appropriate for yeast, any marker gene can be used which facilitates the selection for transformants due to the phenotypic expression of the marker gene. Suitable markers for yeast are, for example, those conferring resistance to antibiotics G418, hygromycin or bleomycin, or provide for prototrophy in an auxotrophic yeast mutant, for example the URA3, LEU2, LYS2, TRP1, or HIS3 gene.

Since the replication of vectors is conveniently done in E. coli, an E. coli genetic marker and an E. coli origin of replication are advantageously included. These can be obtained from E. coli plasmids, such as pBR322, Bluescript© vector or a pUC plasmid, e.g. pUC18 or pUC19, which contain both E. coli replication origin and E. coli genetic marker conferring resistance to antibiotics, such as ampicillin.

Suitable selectable markers for mammalian cells are those that enable the identification of cells competent to take up nucleic acid encoding RNA molecules according to the invention, such as dihydrofolate reductase (DHFR, methotrexate resistance), thymidine kinase, or genes conferring resistance to G418 or hygromycin. The mammalian cell transformants are placed under selection pressure which only those transformants which have taken up and are expressing the marker are uniquely adapted to survive. In the case of a DHFR or glutamine synthase (GS) marker, selection pressure can be imposed by culturing the transformants under conditions in which the pressure is progressively increased, thereby leading to amplification (at its chromosomal integration site) of both the selection gene and the linked DNA that encodes RNA molecules according to the invention. Amplification is the process by which genes in greater demand for the production of a protein critical for growth, together with closely associated genes which may encode a desired protein, are reiterated in tandem within the chromosomes of recombinant cells. Increased quantities of desired protein are usually synthesised from thus amplified DNA.

Expression and cloning vectors usually contain a promoter that is recognised by the host organism and is operably linked to a coding sequence. Such a promoter may be inducible or constitutive. The promoters are operably linked to coding sequences by removing the promoter from the source DNA by restriction enzyme digestion and inserting the isolated promoter sequence into the vector. The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences.

Promoters suitable for use with prokaryotic hosts include, for example, the β-lactamase and lactose promoter systems, alkaline phosphatase, the tryptophan (trp) promoter system and hybrid promoters such as the tac promoter. Their nucleotide sequences have been published, thereby enabling the skilled worker operably to ligate them to coding sequences, using linkers or adaptors to supply any required restriction sites. Promoters for use in bacterial systems will also generally contain a Shine-Delgarno sequence operably linked to the coding sequence.

Preferred expression vectors are bacterial expression vectors which comprise a promoter of a bacteriophage such as phagex or T7 which is capable of functioning in the bacteria. In one of the most widely used expression systems, the nucleic acid encoding the fusion protein may be transcribed from the vector by T7 RNA polymerase (Studier et al, Methods in Enzymol. 185; 60-89, 1990). In the E. coli BL21(DE3) host strain, used in conjunction with pET vectors, the T7 RNA polymerase is produced from the λ-lysogen DE3 in the host bacterium, and its expression is under the control of the IPTG inducible lac UV5 promoter. This system has been employed successfully for over-production of many proteins. Alternatively the polymerase gene may be introduced on a lambda phage by infection with an int- phage such as the CE6 phage which is commercially available (Novagen, Madison, USA). other vectors include vectors containing the lambda PL promoter such as PLEX (Invitrogen, NL), vectors containing the trc promoters such as pTrcHisXpressTm (Invitrogen) or pTrc99 (Pharmacia Biotech, SE), or vectors containing the tac promoter such as pKK223-3 (Pharmacia Biotech) or PMAL (new England Biolabs, MA, USA).

Suitable promoting sequences for use with yeast hosts may be regulated or constitutive and are preferably derived from a highly expressed yeast gene, especially a Saccharomyces cerevisiae gene. Thus, the promoter of the TRP1 gene, the ADHI or ADHII gene, the acid phosphatase (PH05) gene, a promoter of the yeast mating pheromone genes coding for the a- or α-factor or a promoter derived from a gene encoding a glycolytic enzyme such as the promoter of the enolase, glyceraldehyde-3-phosphate dehydrogenase (GAP), 3-phospho glycerate kinase (PGK), hexokinase, pyruvate decarboxylase, phosphofructokinase, glucose-6-phosphate isomerase, 3-phosphoglycerate mutase, pyruvate kinase, triose phosphate isomerase, phosphoglucose isomerase or glucokinase genes, the S. cerevisiae GAL 4 gene, the S. pombe nmt 1 gene or a promoter from the TATA binding protein (TBP) gene can be used. Furthermore, it is possible to use hybrid promoters comprising upstream activation sequences (UAS) of one yeast gene and downstream promoter elements including a functional TATA box of another yeast gene, for example a hybrid promoter including the UAS(s) of the yeast PH05 gene and downstream promoter elements including a functional TATA box of the yeast GAP gene (PH05-GAP hybrid promoter). A suitable constitutive PH05 promoter is e.g. a shortened acid phosphatase PH05 promoter devoid of the upstream regulatory elements (UAS) such as the PH05 (−173) promoter element starting at nucleotide −173 and ending at nucleotide −9 of the PH05 gene.

Gene transcription from vectors in mammalian hosts may be controlled by promoters derived from the genomes of viruses such as polyoma virus, adenovirus, fowlpox virus, bovine papilloma virus, avian sarcoma virus, cytomegalovirus (CMV), a retrovirus and Simian Virus 40 (SV40), from heterologous mammalian promoters such as the actin promoter or a very strong promoter, e.g. a ribosomal protein promoter, and from the promoter normally associated with the structured antisense sequence, provided such promoters are compatible with the host cell systems.

Transcription of a DNA encoding a structured antisense molecule by higher eukaryotes may be increased by inserting an enhancer sequence into the vector. Enhancers are relatively orientation and position independent. Many enhancer sequences are known from mammalian genes (e.g. elastase and globin). However, typically one will employ an enhancer from a eukaryotic cell virus. Examples include the SV40 enhancer on the late side of the replication origin (bp 100-270) and the CMV early promoter enhancer. The enhancer may be spliced into the vector at a position 5′ or 3′ to the coding sequence, but is preferably located at a site 5′ from the promoter.

Advantageously, a eukaryotic expression vector encoding a structured antisense molecule may comprise a locus control region (LCR). LCRs are capable of directing high-level-integration site independent expression of transgenes integrated into host cell chromatin, which is of importance especially where the structured antisense gene is to be expressed in the context of a permanently-transfected eukaryotic cell line in which chromosomal integration of the vector has occurred, in vectors designed for gene therapy applications or in transgenic animals.

Eukaryotic expression vectors will also contain sequences necessary for the termination of transcription and for stabilising the mRNA. Such sequences are commonly available from the 5′ and 3′ untranslated regions of eukaryotic or viral DNAs or cDNAs. These regions contain nucleotide segments transcribed as polyadenylated fragments in the untranslated portion of the mRNA encoding a structured antisense molecule according to the invention.

An expression vector includes any vector capable of expressing nucleic acids that are operatively linked with regulatory sequences, such as promoter regions, that are capable of expression of such DNAs. Thus, an expression vector refers to a recombinant DNA or RNA construct, such as a plasmid, a phage, recombinant virus or other vector, that upon introduction into an appropriate host cell, results in expression of the cloned DNA. Appropriate expression vectors are well known to those with ordinary skill in the art and include those that are replicable in eukaryotic and/or prokaryotic cells and those that remain episomal or those which integrate into the host cell genome. For example, DNAs encoding structured antisense molecules may be inserted into a vector suitable for expression of cDNAs in mammalian cells, e.g. a CMV enhancer-based vector such as pEVRF (Matthias, et al., (1989) NAR 17, 6418).

Construction of vectors according to the invention employs conventional ligation techniques. Isolated plasmids or DNA fragments are cleaved, tailored, and religated in the form desired to generate the plasmids required. If desired, analysis to confirm correct sequences in the constructed plasmids is performed in a known fashion. Suitable methods for constructing expression vectors, preparing in vitro transcripts, introducing DNA into host cells, and performing analyses for assessing Gene product expression and function are known to those skilled in the art. Gene presence, amplification and/or expression may be measured in a sample directly, for example, by conventional Southern blotting, Northern blotting to quantitate the transcription of mRNA, dot blotting (DNA or RNA analysis), or in situ hybridisation, using an appropriately labelled probe which may be based on a sequence provided herein. Those skilled in the art will readily envisage how these methods may be modified, if desired.

In a further aspect, the present invention relates to a host cell transformed with a vector according to the invention has described above.

Preferably, the host cell is a mammalian host cell and may for example be incorporated into an organism. However, the invention also relates to the use of vectors according to the previous aspect thereof for the transformation of cells in order to produce structured antisense molecules. Structured antisense molecules produced in such a manner may be administered to patients and/or organisms by conventional administration techniques.

Host cells such as prokaryote, yeast and higher eukaryote cells may be used for replicating DNA and producing structured antisense molecules. Suitable prokaryotes include eubacteria, such as Gram-negative or Gram-positive organisms, such as E. coli, e.g. E. coli K-12 strains, DH5a and HB101, or Bacilli. Further hosts suitable for structured antisense molecules encoding vectors include eukaryotic microbes such as filamentous fungi or yeast, e.g. Saccharomyces cerevisiae. Higher eukaryotic cells include insect and vertebrate cells, particularly mammalian cells, including human cells, or nucleated cells from other multicellular organisms. In recent years propagation of vertebrate cells in culture (tissue culture) has become a routine procedure. Examples of useful mammalian host cell lines are epithelial or fibroblastic cell lines such as Chinese hamster ovary (CHO) cells, NIH 3T3 cells, HeLa cells or 293T cells. The host cells referred to in this disclosure comprise cells in in vitro culture as well as cells that are within a host animal.

DNA may be stably incorporated into cells or may be transiently expressed using methods known in the art. Stably transfected mammalian cells may be prepared by transfecting cells with an expression vector having a selectable marker gene, and growing the transfected cells under conditions selective for cells expressing the marker gene. To prepare transient transfectants, mammalian cells are transfected with a reporter gene to monitor transfection efficiency.

To produce such stably or transiently transfected cells, the cells should be transfected with a sufficient amount of structured antisense molecules-encoding nucleic acid to form structured antisense molecules. The precise amounts of DNA encoding structured antisense molecules may be empirically determined and optimised for a particular cell and assay.

Host cells are transfected or, preferably, transformed with the above-captioned expression or cloning vectors of this invention and cultured in conventional nutrient media modified as appropriate for inducing promoters, selecting transformants, or amplifying the genes encoding the desired sequences. Heterologous DNA may be introduced into host cells by any method known in the art, such as transfection with a vector encoding a heterologous DNA by the calcium phosphate coprecipitation technique or by electroporation. Numerous methods of transfection are known to the skilled worker in the field. Successful transfection is generally recognised when any indication of the operation of this vector occurs in the host cell. Transformation is achieved using standard techniques appropriate to the particular host cells used.

Incorporation of cloned DNA into a suitable expression vector, transfection of eukaryotic cells with a plasmid vector or a combination of plasmid vectors, each encoding one or more distinct genes or with linear DNA, and selection of transfected cells are well known in the art (see, e.g. Sambrook et al. (1989) Molecular Cloning: A Laboratory Manual, Second Edition, Cold Spring Harbor Laboratory Press).

Transfected or transformed cells are cultured using media and culturing methods known in the art, preferably under conditions, whereby structured antisense molecules encoded by the DNA is expressed. The composition of suitable media is known to those in the art, so that they can be readily prepared. Suitable culturing media are also commercially available.

In a still further aspect of the present invention, vectors as described above may be used in gene therapy techniques and applied to the treatment of diseases. For example, a nucleic acid sequence encoding a structured antisense molecule according to the present invention may be inserted into a viral or non- viral vector designed for the delivery of nucleic acids to the cells of a patient, either ex-vivo or in vivo.

Examples of viral vectors include adenovirus vectors, adenoassociated virus vectors, retroviral vectors. Examples of non- viral vectors include naked DNA, condensed DNA particles, liposome-type vectors which may include a targeting moiety and, if applicable, escape peptides derived from viruses, and DNA complexed to targeting moieties such as antibodies or cell surface ligands, which are preferably internalised by the target cell.

Alternatively, however, structured antisense molecules according to the invention may be delivered by conventional medicinal approaches, in the form of a pharmaceutical composition. A pharmaceutical composition according to the invention is a composition of matter comprising the combination of a structured antisense molecule as an active ingredient. The active ingredients of a pharmaceutical composition according to the invention are contemplated to exhibit excellent therapeutic activity, for example, in the alleviation of diseases involving the expression of an aberrant RNA molecule. Dosage regima may be adjusted to provide the optimum therapeutic response. For example, several divided doses may be administered daily or the dose may be proportionally reduced as indicated by the exigencies of the therapeutic situation.

The active compound may be administered in a convenient manner such as by the oral, intravenous (where water soluble), intramuscular, subcutaneous, intranasal, intradermal or suppository routes or implanting (e.g. using slow release molecules). Depending on the route of administration, the active ingredient may be required to be coated in a material to protect said ingredients from the action of enzymes, acids and other natural conditions which may inactivate said ingredient.

In order to administer the combination by other than parenteral administration, it will be coated by, or administered with, a material to prevent its inactivation. For example, the combination may be administered in an adjuvant, co-administered with enzyme inhibitors or in liposomes. Adjuvant is used in its broadest sense and includes any immune stimulating compound such as interferon. Adjuvants contemplated herein include resorcinols, non-ionic surfactants such as polyoxyethylene oleyl ether and n-hexadecyl polyethylene ether. Enzyme inhibitors include pancreatic trypsin.

Liposomes include water-in-oil-in-water CGF emulsions as well as conventional liposomes.

The active compound may also be administered parenterally or intraperitoneally. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions (where water soluble) or dispersions and sterile powders for the extemporaneous preparation of sterile injectable solutions or dispersion. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene gloycol, and the like), suitable mixtures thereof, and vegetable oils. The proper fluidity can be maintained, for example, by the use of a coating such as lecithin, by the maintenance of the required particle size in the case of dispersion and by the use of superfactants.

The prevention of the action of microorganisms can be brought about by various antibacterial and antifungal agents, for example, parabens, chlorobutanol, phenol, sorbic acid, thirmerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compound in the required amount in the appropriate solvent with various of the other ingredients enumerated above, as required, followed by filtered sterilisation. Generally, dispersions are prepared by incorporating the sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the combination of polypeptides is suitably protected as described above, it may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. The amount of active compound in such therapeutically useful compositions in such that a suitable dosage will be obtained.

The tablets, troches, pills, capsules and the like may also contain the following: a binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier.

Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound may be incorporated into sustained-release preparations and formulations.

As used herein “pharmaceutically acceptable carrier and/or diluent” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the novel dosage unit forms of the invention are dictated by and directly dependent on (a) the unique characteristics of the active material and the particular therapeutic effect to be achieved, and (b) the limitations inherent in the art of compounding such as active material for the treatment of disease in living subjects having a diseased condition in which bodily health is impaired.

The principal active ingredients are compounded for convenient and effective administration in effective amounts with a suitable pharmaceutically acceptable carrier in dosage unit form. In the case of compositions containing supplementary active ingredients, the dosages are determined by reference to the usual dose and manner of administration of the said ingredients.

In a further aspect there is provided the structured antisense molecule as hereinbefore defined for use in the treatment of disease. Consequently there is provided the use of a combination of the invention for the manufacture of a medicament for the treatment of disease associated with expression of aberrant RNA. The aberrant nature of the RNA may be due to chromosomal translocation.

The invention is described below, for the purposes of illustration only, in the following examples.

### EXAMPLE 1

### Preparation of Structured Antisense Molecules

The BCR-ABL fusion mRNA results from the translocation t(9;22), which is found in CML and ALL, encoding p210 and p190 proteins respectively (13, 14). The difference between the BCR-ABL protein sizes reflects differences in the breakpoints within the BCR gene (15) resulting in two distinct mRNAs containing the same ABL exons fused to different BCR sequences. Thus the CML and ALL BCR-ABL fusion mRNA junctions differ only on one side. This provides a model target for analysis of structured antisense RNA interactions. BCR-ABL antisense RNAs are designed with the aid of the M-FOLD programme (16, 17, 18). These molecules have the potential to fold spontaneously on synthesis into a double hairpin structure, with a short stretch of single strand between the two hairpins (FIG. 1A.). This single stranded region (the targeting region) is complementary to the 7 bases of 3′ sequence (in this case ABL) immediately adjacent to the fusion junction of the BCR-ABL p190 mRNA and 1 base of sequence 5′ of the junction. The remaining region of antisense (designated stem/loop II; ascending strand) continues the sequence complementarity to BCR 5′ of the fusion junction for another 31 residues. The descending sequence of stem/loop II (black, thin line in FIG. 1A) base pairs with the antisense sequence (this differs in the indicated places in two forms of hAS190 which we have designed, α and β respectively, FIG. 1A). The 5′ stem/loop (stem/loop I, FIG. 1A), which in both hAS190 α and β forms contains antisense sequence to ABL, is required primarily to initiate and stabilise the desired folding of the molecule and can be formed from completely irrelevant sequence without affecting function.

The hAS structured antisense RNAs are made by cloning synthetic oligonucleotides adjacent to a T7 promoter in a plasmid vector, and have the sequences indicated herein. RNAs are prepared by run-off in vitro transcription. A plasmid containing the hAS190 sequence is linearised with XbaI and transcribed from the T7 promoter using commercially available kits. The RNA is ethanol-precipitated twice from 0.5M ammonium acetate to remove free nucleotides and then dephosphorylated using 2 u calf intestinal alkaline phosphatase (Boehringer-Mannheim) per microgram of RNA. The enzyme is removed by the addition of 0.5% SDS and 10 μg/ml proteinase K, and incubating at 37° C. for 20 min., followed by extraction with phenol/chloroform/iso-amyl alcohol and ethanol-precipitation twice from 0.3M sodium acetate. Finally the RNA is end labelled using 1 u/μg polynucleotide kinase (NEB) and 50μCi γ³²P-ATP (Amersham), for 1 hour at 37° C.

The spontaneous folding of in vitro transcripts is assessed by partial T₁ ribonuclease digestion in which preferential cleavage at G residues is used as an assessment of predicted structure since these residues should be resistant to T₁ ribonuclease within double-stranded regions. End labelled RNA is digested with 25 u/ml T1 ribonuclease in sequencing buffer (8.3M urea; 25 mM sodium citrate pH 3.5; 1.5 mM EDTA) at 55° C. for 15 min., to cleave after every G residue, or in 300 mM NaCl; 10 mM Tris-HCl pH 7.5; 5 mM EDTA at 37° C. for 15 min. to cleave G residues exposed in the native structure. The ladder is generated by partial alkaline hydrolysis of the transcript (50 mM NaPO₄, pH12; 55° C., 15 min.). Fragments are separated on a 7% polyacrylamide, 8M urea sequencing gel, run at 20V/cm.

The pattern of bands found by T₁ ribonuclease cleavage of hAS190α (FIG. 1B, lane 2) shows almost complete T₁ ribonuclease-resistance of the stem/loop II region, except for two G's close to the targeting region (presumably susceptible due to breathing of the basal region). These data are consistent with the structure predicted by the M-Fold programme, which is used to design the hAS molecules.

### EXAMPLE 2

### Binding of Structured Antisense Molecules to Target

The ability of hAS190 transcripts to bind a p190 BCR-ABL target RNA (generated by in vitro transcription of a cloned p190 BCR-ABL cDNA fragment) is shown in FIG. 2. Samples are applied to a continuously running gel after hybrid formation at 37° C. for the different times indicated.

Target RNAs are transcribed using standard kit protocols from the T3 or T7 promoters of pBluescriptII, into which had been cloned the BglIH-KpnI fragment of p190 BCR-ABL (p190(+) or (−)); the HindIII-KpnI fragment of p210 BCR-ABL (p210(+)); the NarI-KpnI fragment of the ABL-b isoform (ABL(+)) or the BamHI-SalI fragment of BCR. (BCR(+)). Yeast tRNA is used as a control of non-relevant RNA and is included in all reactions at 0.5 mg/ml as a carrier.

Incubation of ³²P-labeled hAS190α or β with ³²P-labeled target fusion mRNA (p190(+) RNA) over 120 minutes at 37° C. followed by native gel electrophoresis, results in the appearance of a species which co-migrates with a sense-antisense hybrid produced by denaturing the RNAs and annealing at 65° C. (FIG. 2A, An). Approximately 2 pmol of the p190 mRNA fragment (p190(+)RNA) is mixed with hAS190α (2 pmol) or hAS190β (10 pmol) antisense in reaction buffer at 37° C.

In general, RNA interactions are carried out at 37° C. in reaction buffer (250 mM NaCl; 10 mM Tris-HCl, pH 8; 0.5 mg/ml yeast tRNA). Reaction volumes are 10 μl except for the time series (FIG. 2) where 5 μl aliquots are taken from a 40 μl reaction. Reactions are stopped by mixing with an equal volume of ice-cold glycerol buffer (40% glycerol; 20 mM Tris-HCl, pH8; 10 mM EDTA) or loading buffer (95% formamide; 5 mM EDTA, pH8), for native and denaturing gels respectively. Samples for native gels are loaded on gels and run immediately. Samples in loading buffer can be stored on ice for several hours prior to electrophoresis, then either loaded directly on part-denaturing (4M urea) gels or heated at 95° C. for 5 minutes before loading on denaturing (8M urea) gels.

At the times indicated in FIG. 2, aliquots are removed and immediately loaded on a continuously electrophoresing 5% native polyacrylamide gel. Each sample is run into the gel for 2 min. at 10V/cm then the voltage is reduced to 1V/cm until the next sample is loaded. After the last sample had been loaded, the gel is electrophoresed to completion at 10V/cm, then fixed, dried and exposed to X-ray film. This hybridisation product appears within 10 minutes at 37° C. and by 120 minutes, and essentially all the sense RNA is hybridised. The rate of appearance of the hAS190α/p190(+) hybrid appears to be significantly faster than the hAS190β/p190(+) hybrid. This is consistent with the rate of interaction being determined by the change in free energy when transforming from structured antisense to antisense-target hybrid. The hAS190β molecule has a more stable stem/loop II structure than hAS190α, resulting in a smaller −ΔG on hybridisation, and should therefore interact more slowly with the target.

The presence of both RNA molecules in the new hybrid RNA species is formally proven by gel analysis of hybridisation products after incubating, at 37° C., either labelled hAS190α with cold p190 (+) RNA (FIG. 2B, lane 2), cold hAS190α with labelled p190 (+) RNA (FIG. 2B, lane 3) or both labelled RNAs (FIG. 2B, lane 1). A similar protocol to that of FIG. 2A is repeated using both ³²-P radio-labelled and unlabelled hAS190α and p190(+) RNAs. The lanes shown are the 30 min. reactions at 37° C. The hybrid generated by hAS190α appears as a doublet probably as a result of weak interactions involving the stem/loop II sequences, since mildly denaturing conditions abolish the upper band of the doublet completely (FIG. 3). In addition doublet formation appears to be peculiar to the hAS190α form since hAS190β, which differs only in a few bases in the descending strand of hairpin II (FIG. 1A.), makes a single hybrid species (FIG. 2A.).

### EXAMPLE 3

### Specificity of Binding

The foregoing data show that simple incubation conditions promote hybrid formation between the p190 mRNA target and the hAS190 structured antisense. The specificity of this association is tested by preparing in vitro transcripts corresponding to fragments of p210 BCR-ABL, ABL and BCR mRNAs (n2), all of which share some sequence with the sense strand of p190 BCR-ABL (the p210 form of BCR-ABL having identical ABL but different BCR sequences and thus has a distinct junctional sequence). These RNAs are used in 37° C. hybridisation reactions, in conditions of large antisense excess, with either the hAS190α or hAS210 (a structured antisense which is designed to bind the p210 BCR-ABL). 2 pmol ³²P-labeled hAS190α or hAS210 (The sequence of the hAS210 RNA is: 5′-GGGCGAAUUGGAUUCGCCCGGGCUUUUGAACUCUGCUU AAAUCCAGUGGCUGAGUGGAUCUUCCACUUAGCUACUGGACUUAAGUAGU GUUCAUGCAUCUAG-3′) is mixed with 0.2 pmol of the indicated unlabelled target RNA species as described in Example 2 and allowed to associate at 37° C. in reaction buffer as indicated in the foregoing example for general RNA reactions. Reactions are stopped by the addition of formamide loading buffer and kept on ice until they are loaded onto a 5% polyacrylamide gel containing 4M urea. The gel is run at 10V/cm, fixed, dried and exposed to film for 1 hour. mRNA fragments are designated p190(+), p210(+), ABL(+), BCR(+) and an opposite (antisense) fragment is designated p190(−).

These hybridisation reactions only yield productive hybrid when the structured antisense is incubated with its cognate RNA target (FIG. 3): Antisense hAS190α only formed a hybrid with p190 RNA and did not hybridise with p210, ABL or BCR RNAs, whilst hAS210 hybridises to p210 RNA only (overnight exposures, or running samples on native gels, using glycerol loading buffer, also failed to show hybrid bands appearing in any of the control lanes). Thus the results of the hybridisation experiments demonstrate both efficacy and specificity of the structured antisense molecules in vitro.

### EXAMPLE 4

### Blocking of RNA Unwinding

A model for the nucleation-unwinding and hybrid formation is shown in FIG. 1C. Experimental data supporting the proposed model are obtained by blocking experiments carried out with oligonucleotides complementary to three regions of the hAS190α antisense molecule (shown in FIG. 4B). Blocking oligonucleotides are 15- or 16-mers with similar predicted Tm (48-50° C.). The sequences are: oligo 1 5′AGACGCA GAAGCCCG; oligo 2 5′GTAGAACGATGGCGAG; oligo 3 5′GGCGCCTTCCA TGGA. ³²P-labeled hAS190α (1 pmol) is mixed with the indicated concentration of oligonucleotide in reaction buffer (as described in Example 2) and incubated at room temperature for 2 min. Unlabelled mRNA fragment p190(+) RNA (1 pmol) is then added and the reaction incubated at 37° C. for 30 min. The reactions are run on a 5% native polyacrylamide gel prior to autoradiography. Only oligo 1, which is complementary to the targeting region of hAS190α, suppresses the interaction of hAS190α with its p190(+) target (FIG. 4A). Oligonucleotides 2 and 3, the latter including the region at the apex of stem loop II, did not inhibit hybrid formation. Thus the initial hybridisation does seem to occur at the base of stem/loop II.

### EXAMPLE 5

### Inhibition of BCR-ABL Expression In Vivo

Introduction of Pre-Formed RNA With Target Gene Expression Vectors in COS-7 Cells

In order to determine the capacity of structured antisense RNAs (hAS) to interact with and block the expression of a target niRNA in the cells, COS-7 cells are cotransfected with an expression construct containing the BCR-ABL p190 cDNA behind an SV 40 promoter, and the hAS 190 RNA in its native form. This allows assessment of inhibition of BCR-ABL expression, after de novo synthesis, in the presence of the blocking antisense RNA.

hAS 190 is synthesised in vitro as previously described. Transfection is effected using the proprietary reagent Superfect (Quiagen) which efficiently transduces both RNA and DNA into cells. A large molar excess of RNA (between 200 and 400 fold) is used to compensate for the production of high level transcripts from each translated expression plasmid. Translated cells are cultured for 18-24 hours, which allows for the detection of BCR-ABL protein in the control population. The cells are then harvested and prepared for analysis of the protein constituents by SDS-PAGE.

Production of BCR-ABL is detected by Western blotting using monoclonal antibodies to BCR and ABL proteins. As controls, yeast tRNA and hAS RNAs of different specificities are cotransfected with the p190 BCR-ABL expression construct into COS-7 cells. Similarly, hAS 190 with is cotransfected P 210 BCR-ABL, BCR or ABL cDNAs driven from identical promoters, all of which can be distinguished from one another and from indigenous cellular homologues by the monoclonal antibodies used for the Western blotting analysis.

Introduction of hAS Expression Vectors with Target Gene Expression Vectors in COS-7 Cells

The expression vectors used in this experiment are required to generate useful levels of hAS RNA in cells on a stable basis. As the hAS RNAs are required to fold spontaneously on synthesis into their intended configuration expression cassettes need to give moderate to high levels of transcription and result in minimal addition of promoter/terminator-encoded sequence appended to the hAS 5′ or 3′ ends.

This is achieved using two strategies based on a pol II or pol III promoters. A normal pol II promoter requires minimal 5′ sequence to be promoter-specified, especially if an initiator sequence is inserted downstream of the TATA box to more clearly define the transcription start site. However, for optimal expression levels, considerable 3′ sequence is required including a polyadenylation signal and possibly a spliceable intron. However these requirements are overcome by the introduction of a ribozyme sequence immediately downstream of the hAS encoding region, which precisely cleaves the nascent RNA at the end of the hAS sequence.

The second strategy uses pol III promoters which are used to synthesise small RNAs in vivo. Most pol III promoters require specific gene-internal sequences to function, however the U6 snRNA promoter is entirely self-contained, has a well defined transcription start signal and terminates on encountering an oligo-T element in the gene sequence. Potential problems with this promoter are its potential turnover rates and the inability to produce hAS RNAs containing a sequence of more than four consecutive U residues.

Both types of vector are used to coexpress hAS RNAs alongside the expression vectors described in the previous section, using similar transfection and detection protocols.

Results

Using both experimental protocols outlined above, production of target mRNA and protein is observed to be either significantly decreased or, in some cases, entirely absent in the presence of hAS190 RNA. In contrast, its production is not inhibited in the control experiments.

### EXAMPLE 6

In a further in vivo experiment, HeLa cells are transfected with vectors encoding BCR-ABL p190 or p210 proteins, together with a vector encoding the hAS-190α structured antisense RNA. The vectors used in this experiment are constructed as follows:

PUN1

The human U6 snRNA promoter sequence is amplified from genomic DNA using Pfu polymerase in a PCR reaction. Utilising restriction sites embedded in the primers, the 328 bp promoter region is cloned into (XhoI+EcoRV cut) pBluescript_{II} SK(+) as an XhoI:blunt fragment. The polIII terminator cassette is cloned as annealed synthetic oligonucleotides into XbaI and SacI-cut plasmid, destroying the SacI site in the process.

PUN1.SQ2c1 is derived by subcloning a blunt ended PstI+EcoRI fragment of pSK(+).SQ2c1 (encoding the hAS190α RNA) into pUN1, cut with SacI and polished with T4 polymerase. The plasmid pSK(+).SQ2c1 is constructed as described, by inserting synthetic oligonucleotides encoding hAS 190α the EcoRV site of pBluescript.

PUN1.Tal1 is derived by subcloning an EcoRI+KpnI (polished) fragment from pGEM4.Tal1.1 (incorporating sequence from exon 6 of the Tal1 gene) into pUN1, cut with EcoRI+PstI (polished).

For transfection, HeLa cells are seeded into 6 well plates at 2×10⁵ cells/well to give a 50% confluent monolayer the following day.

For each well:

100 μl OptiMEM (Gibco-BRL) is placed into a sterile Eppendorf with a total of 4 μg of plasmid DNA for each individual transfection (a single well). Plasmid ratios are 0.8:3.2 μg for pE1A2:pUN1(x) and 1.2:2.8 μg for pKW3:pUN1(x) transfections, giving approximate molar ratios of 1:8 (reporter:antisense) in each case. Plasmid pE1A is a cloning vector (pCDX) comprising the p190 4.5 kb cDNA cloned at the EcoR1 site; plasmid pKW3 is a vector (pCDX-neo) comprising the 6.7 kb p210 cDNA cloned at the EcoR1 site. The vectors express p190 and p210 respectively.

10 μl Superfect (Qiagen) is added to each tube and the contents vortexed briefly. The tubes are allowed to stand at room temp for 5 min. to permit complex formation.

Growth medium is then aspirated from the cells.

0.5 ml prewarmed complete medium (Dulbecco's modified Eagle's medium, including 10% foetal calf serum and antibiotics) is added to each Eppendorf and the contents mixed by pipetting up and down twice, avoiding air bubbles. The resulting mixture is added to the cell monolayer, ensuring even coverage. The cells are then placed in a gassed incubator for 3 hours.

The medium+complexes are then aspirated and replaced with 2 ml complete medium, before returning the cells to the incubator overnight.

Western blot analysis of transfected HeLa cells

The following day the medium is aspirated and the monolayer washed with 2 ml PBS/EDTA. 0.3 ml trypsin (in PBS/EDTA) is added to each well and the plates incubated at 37° C. for 2-5 min. until the cells start to detach. 1 ml of complete medium is added to each well to inactivate trypsin, and the cells detached thoroughly by pipetting up and down. The suspended cells are transferred to a 1.5 ml Eppendorf and spun at 6500 rpm for 3 min. in a benchtop microfuge. All traces of medium are carefully aspirated with a vacuum line and the cells resuspended thoroughly in 20 μl PBS/EDTA. 30 μl 2×SDS sample buffer is added, and the tube vortexed briefly before being placed in a boiling waterbath for 5 min. The lysates are spun briefly at 15 k, vortexed again to remix the sample and chilled on ice before loading on a 5-15%SDS-PAGE gel (5-15 μl per well).

Remaining sample is stored at −20° C.

After electrophoresis, proteins are transferred to a PVDF membrane using an electroblotting apparatus and probed with either a monoclonal anti-cABL (8E9, used at 1:400 dilution of crude tissue culture supernatant) or a rabbit polyclonal IgG anti-BCR (Santa Cruz Biotech. Inc., used at 1:2,000 dilution). Detection is by ECL (Amersham) using standard protocols. Levels of protein are quantitated at various times after transfection.

Results obtained 16 hours post-transfection are shown in FIGS. 5 and 6. In FIG. 5, HeLa cells are cotransfected with p190 and hAS-p190α. As controls, the empty vector and a vector encoding non-relevant antisense against the TAL-1 mRNA are included. A reduction in p190 protein levels of approximately 50% is observed, representing a significant reduction in protein levels. Native BCR protein levels, in contrast, are unaffected.

In FIG. 6, a control experiment is shown wherein HeLa cells are cotransfected with p210 and hAS-p190α. Although p210 expression is weak compared to p190 expression as shown in FIG. 5, it is nonetheless apparent that there is little or no reduction in p210 levels. This confirms the specificity of hAS-p190α.

### REFERENCES

1. T. H. Rabbitts, Nature 372, 143-149 (1994).

2. T. Rabbitts, N Engl J Med 338, 192-4 (1998).

3. R. Kurzrock, J. U. Gutterman, M. Talpaz, N. Engl. J. Med. 319, 990-998 (1988).

4. A. Kabisch, L. Perenyi, U. Seay, J. Lohmeyer, H. Pralle, Acta Haematol 92, 190-6 (1994).

5. S. O'Brien, et al., Leukemia 8, 2156-62 (1994).

6. T. Skorski, et al., Proc Natl Acad Sci U S A 91, 4504-8 (1994).

7. T. Smetsers, et al., Leukemia 8, 129-40 (1994).

8. T. Smetsers, et al., Leukemia 9, 118-30 (1995).

9. T. Smetsers, E. Mensink, Blood 85, 597-8 (1995).

10. R. Kronenwett, R. Haas, G. Sczakiel, J Mol Biol 259, 632-44 (1996).

11. D. Herschlag, Proc. Natl. Acad. Sci. USA 88, 6921-6925 (1991).

12. T. Woolf, D. Melton, C. Jennings, Proc Natl Acad Sci U S A 89, 7305-9 (1992).

13. A. Hermans, et al., Cell 51, 33-40 (1987).

14. E. Shtivelman, B. Lifshitz, R. P. Gale, E. Canaani, Nature 315, 550-554 (1985).

15. J. Groffen, et al., Cell 36, 93-99 (1984).

16. M. Zucker, Science 244, 48-52 (1989).

17. J. A. Jaeger, D. H. Turner, M. Zucker, Proc. Natl. Acad. Sci. (USA) 86, 7706-7710 (1989).

18. J. A. Jaeger, D. H. Turner, M. Zucker, in Molecular Evolution: Computer Analysis of Protein and Nucleic Acid Sequence R. F. Doolittle, Eds. 1989), vol. 183, pp. 281-306.

1. An antisense nucleic acid molecule comprising a first region and a second region, both of which are complementary to a target nucleic acid molecule, and wherein the first region is available for hybridisation and the second region is temporarily masked.

2. The antisense molecule according to claim 1, wherein the second region is temporarily masked through being comprised in a hairpin loop structure.

3. The antisense molecule according to claim 1 or claim 2, wherein the first region is complementary to a specific sequence of the target nucleic acid molecule.

4. The antisense molecule according to claim 1, wherein the first region is between three and 18 bases in length.

5. The antisense molecule according to claim 1, wherein the first region exists in a single-stranded form.

6. The antisense molecule according to claim 1, wherein the second region is complementary to a sequence of the target nucleic acid contiguous with the specific sequence complementary to the first region.

7. The antisense molecule according to claim 1, wherein the unmasking of the second region becomes energetically favourable only in the presence of the target nucleic acid molecule and after interaction therewith via the first region.

8. The antisense molecule according to claim 2, wherein the hairpin loop comprising the second region contains one or more destabilising elements.

9. The antisense molecule according to claim 1, wherein the second region is no longer than 100 bases in length.

10. The antisense molecule according to claim 1, which is RNA or DNA.

11. The antisense molecule according to claim 1, wherein the target nucleic acid is RNA or DNA.

12. A method for hybridising an antisense nucleic acid molecule to a target nucleic acid, comprising the steps of:
(a) preparing an antisense nucleic acid molecule according to claim 1;
(b) hybridising the antisense molecule of step (a) to the target nucleic acid such that the first region of the antisense molecule binds to its complementary sequence in the target nucleic acid, such that the second region of the antisense molecule hybridises to its complementary sequence in the target nucleic acid.

13. A method for modulating the expression of a gene product encoded by a target nucleic acid, comprising the steps of:
(a) preparing an antisense nucleic acid molecule according to claim 1;
(b) hybridising the antisense molecule of step (a) to the target nucleic acid comprising a gene encoding said gene product such that the first region of the antisense molecule binds to its complementary sequence in the target nucleic acid, such that the second region of the antisense molecule hybridises to its complementary sequence in the target nucleic acid. Wherein said hybridising of said antisense molecule to said target nucleic acid permits modulation of expression of gene.

14. The method according to claim 12 or claim 13, wherein the second region of the antisense nucleic acid molecule is temporarily masked in a hairpin loop structure.
